(19) United States
(12) Patent Application Publication
(10) Pub. No.: US 2017/0112823 A1
(43) Pub. Date: Apr. 27, 2017

(54) COMPOSITIONS AND METHODS TO TREAT NEUROPATHY AND RELATED CONDITIONS

(71) Applicant: Health Naturally, LLC, Rancho Santa Margarita, CA (US)

(72) Inventor: Alok Nigam, Trabuco Canyon, CA (US)

(73) Assignee: Health Naturally, LLC, Rancho Santa Margarita, CA (US)

(21) Appl. No.: 15/297,928

(22) Filed: Oct. 19, 2016

Related U.S. Application Data
(60) Provisional application No. 62/247,093, filed on Oct. 27, 2015.

(51) Int. Cl. A61K 31/4745; A61K 31/355; A61K 36/324; A61K 45/06; A61K 31/121; A61K 31/4525

(52) U.S. Cl. CPC A61K 31/4745; A61K 31/121; A61K 31/4525; A61K 36/324; A61K 45/06; A61K 31/355

(57) ABSTRACT

In various embodiments, the invention relates to compositions and methods for treating a condition associated with inflammation. In some embodiments the condition may include neuropathy or arthritis. In various embodiments, the invention teaches kits for treating a condition associated with inflammation. In some embodiments, the invention teaches kits for treating neuropathy or arthritis.

### CROSS-REFERENCE TO RELATED APPLICATION

This application claims the benefit under 35 U.S.C. §119(e) of U.S. Provisional Patent Application No. 62/247,093 filed on Oct. 27, 2015, which is incorporated herein by reference in its entirety.

### FIELD OF THE INVENTION

The invention relates to compositions, methods and kits for treating neuropathy and related conditions.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Nerves are the communication lines in the body. Electrical signals from the brain travel through the nerves and give instructions to the various body parts. The body receives information from the environment through the senses and sends this information to the brain via the nerves. Nerve cells have an outer sheathing or myelin protective covering (FIG. 1A). Myelin sheath is made up of up to 75-80% cholesterol and lipids (fats). It can be damaged by aggressive management of cholesterol and a very low fat diet. It can also be degraded by an excessive amount of free radicals and compression on the nerves. With nerve damage, the outer sheathing or myelin protective covering of nerve cells degenerates. Without this protection, the electrical signals are not transferred properly (FIG. 1B). As the nerve damage gets worse, the nerves either lose their ability to transmit information (numbness), or they start sending false signals (pain and tingling). Neuropathy is a condition where the nerves have sustained enough damage that there is noticeable numbness, pain or tingling.

Peripheral neuropathy (PN) develops when nerves that carry messages to and from the brain and spinal cord to the rest of the body are damaged, diseased, inflamed or malfunction resulting in distorted signals. It more often affects the limb extremities. Symptoms of PN include: burning, stabbing or shooting pain; pain or loss of ability to feel pain; tingling, numbness and swelling; pins and needle sensation and hypersensitivity; coldness; and foot and leg ulcer infection and gangrene.

PN can be caused by poor nutrition, diseases (including diabetes and cancer), pressure or trauma and toxins. 30% of neuropathies are “idiopathic” or of unknown cause. Causes of PN can include: diabetes; vitamin deficiencies, especially B12, folate, B1 and other B vitamins; inflammation; oxidative stress and excessive free radicals; poor blood circulation, oxygen deficiency; accumulated toxins due to poor functioning excretory system, or excessive intake by food; biological imbalance as in kidney or liver disease; cancer and chemotherapy; injuries, trauma, surgery, or repetitive use damage, such as carpal tunnel syndrome; infections such as HIV, Herpes, Shingles, and Lyme disease; excessive alcohol use; and very low fat diets. Various drugs including, chemotherapy drugs, BP meds, statins, some anti-viral treatments specifically for HIV can cause PN.

Prevalence of PN in US is estimated to be 20 million. More than 50% of diabetics have some form of neuropathy. In diabetic peripheral neuropathy (DPN), high blood glucose levels damage tiny blood vessel that supply oxygen and nutrients to the nerves of the hands and feet, and to the essential organs like the eyes, kidney, and heart. Poor blood supply and hence lack of nutrients and oxygen cause DPN (FIGS. 2A-2B). Both diabetes and neuropathy are on the rise. US Medicare spends over $3.5 billion on neuropathy care.

PN care is provided by a variety of professionals including internal medicine doctors, neurologists, spine specialists, podiatrists, endocrinologists, rheumatologists, pain medication specialists, chiropractors, and cardiologists after heart surgery. Common PN treatments include: medications such as anti-inflammatory drugs, physical therapy, rest, hot and cold, cool laser treatment, ultrasound, corticosteroid injections or oral steroids, occasional surgery to fix nerve compression, such as carpal tunnel syndrome, spine and lower back, sometimes amputation specifically of lower legs, and acupuncture

Medications used for management of PN are from three general groups: epilepsy drugs, antidepressants, and opioids. They all have significant side effects. Epilepsy drugs include Pregabalin (LYRICA), Gebapentin, and Carbamazepine (Tegretol and Carbatrol). For example, LYRICA side effects include: very commonly (>10%), dizziness and drowsiness; and commonly (1-10%), blurred vision, diplopia, weight gain, vivid dream, confusion, change in libido, memory impairment, vertigo, erectile dysfunction, vomiting, flatulence, dry mouth, constipation, nausea, depression, flushing, rash, and muscle cramp etc. Antidepressants include Venlafaxine (Effexor), Duloxetine (Cymbalta), and various tricyclic antidepressants. For example, Cymbalta side effects include: nausea (34%), dry mouth (23%), headache (20%), dizziness (19%), reduced libido, suicidal thoughts, and difficult to get off the drug. Opioids include Oxycodone (OxyContin and Oxecta), and Tramedol (Conzip and Ultram). For example, opioids have standard side effects associated with narcotics, including addiction and dependency.

Complementary medicines and supplements include: marijuana (cannabis), Metanx, and Neurobion. Marijuana (cannabis) acts as an anti-depressant, is a narcotic drug and can be addictive. It down regulates Glutathione, and creates nutritional deficiencies. Metanx is a medical food. Its ingredients include: Vitamin B12, B6, and folic acid, but it lacks Vitamin B1 and B2 essential for myelin sheath development and accelerated healing. Neurobion is a combination of B1, B6, and B12. It is sold as an over-the-counter (OTC product), but it lacks Folate. It is water soluble and flushed out before body can utilize them.

None of these treatments address the underlying cause of the disease. They all address the pain symptoms. To get relief, patients have to continue to take medications. With these drugs' side effects, quality of life further deteriorates. Therefore, there is still an unmet need for better PN treatments. There is a similar unmet need for better arthritis treatments.

### SUMMARY OF THE INVENTION

In various embodiments, the invention teaches a composition for treating neuropathy and/or arthritis. In some embodiments, the composition includes vinpocetine and one or more of the following compounds: curcumin, piperine, commiphora mukul extract, boswellia serrata extract, boswellia Acetyl-11-keto-β-boswellic acid (AKBA), and withania somnifera. In some embodiments, the composition includes vinpocetine, curcumin and piperine. In some embodiments, the foregoing compositions further include boswellia serrata extract. In some embodiments, the foregoing compositions further include one or more phospholipids selected from the group consisting of phosphatidylcholine, phosphatidic acid, phosphatidylethanolamine, and phosphatidylserine. In some embodiments, the foregoing compositions further include one or more compound selected from the group consisting of Vitamin C, Vitamin B1, Benfotiamine, Vitamin B2, Vitamin B3, Vitamin B6, Methylcobalamin B12, L-taurine, alpha-lipoic acid and Folic Acid. In some embodiments, the foregoing compositions further include Vitamin E and/or Vitamin E Acetate. In some embodiments, the foregoing compositions further include one or more of the following: water, sesame oil, mineral oil, vegetable oil, Cetearyl Alc. & Cetearyl Gluco., Cetearyl Alcohol, Glyceryl & PEG-100 Stearate, Menthol, Glycerin Monolaurate, Propylene Glycol, Glycerin, Cetyl Myristoleate, Cetyl alcohol, Dimethicone, Shea Butter, Winter green oil, Aloe 10X, Polyhexameth. Biguanide, Cyclopentasiloxane, Ascorbyl Palmitate, Ethyl Lauryl Arginate, Ultrez 20 Carbomer, BHA, and Dehydro Acetic Acid. In some embodiments, the foregoing compositions include glycerin monolaurate. In some embodiments, the foregoing compositions include cetyl myristoleate. In certain embodiments, the foregoing compositions include L-Taurine.

In various embodiments, the invention teaches a method for treating a condition associated with inflammation. In some embodiments, the method includes providing a composition that includes vinpocetine and one or more of the following compounds: curcumin, piperine, commiphora mokul extract, boswellia serrata extract, boswellia Acetyl-11-keto-β-boswellic acid (AKBA), and withania somnifera; and administering an effective amount of the composition to the subject, thereby treating the condition associated with inflammation. In certain embodiments, the composition includes vinpocetine, curcumin and piperine. In some embodiments, the foregoing compositions further include boswellia serrata extract. In some embodiments, the foregoing compositions further include one or more phospholipids selected from the group consisting of phosphatidylcholine, phosphatidic acid, phosphatidylethanolamine, and phosphatidylserine. In some embodiments, the foregoing compositions further include one or more compound selected from the group consisting of Vitamin C, Vitamin B1, Benfotiamine, Vitamin B2, Vitamin B3, Vitamin B6, Methylcobalamin B12, L-taurine, alpha-lipoic acid and Folic Acid. In some embodiments, the foregoing compositions further include Vitamin E and/or Vitamin E Acetate. In some embodiments, the foregoing compositions further include one or more of the following: water, sesame oil, mineral oil, vegetable oil, Cetearyl Alc. & Cetearyl Gluco., Cetearyl Alcohol, Glyceryl & PEG-100 Stearate, Menthol, Glycerin Monolaurate, Propylene Glycol, Glycerin, Cetyl Myristoleate, Cetyl alcohol, Dimethicone, Shea Butter, Winter green oil, Aloe 10X, Polyhexameth. Biguanide, Cyclopentasiloxane, Ascorbyl Palmitate, Ethyl Lauryl Arginate, Ultrez 20 Carbomer, BHA, and Dehydro Acetic Acid. In some embodiments, the foregoing compositions include glycerin monolaurate. In some embodiments, the foregoing compositions include cetyl myristoleate. In some embodiments, the foregoing compositions further include L-Taurine. In certain embodiments, the condition associated with inflammation is neuropathy. In some embodiments, the condition associated with inflammation is arthritis. In certain embodiments, the subject is a human. In some embodiments, the composition is administered orally to the subject. In certain embodiments, the composition is administered topically to the subject.

In various embodiments, the invention teaches a kit for treating a condition associated with inflammation in a subject. In some embodiments, the kit includes a composition described above and instructions for using the composition to treat a condition associated with inflammation. In some embodiments, the condition is neuropathy. In certain embodiments, the condition is arthritis.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A illustrates, in accordance with various embodiments of the invention, a healthy peripheral nerve cell.

FIG. 1B illustrates, in accordance with various embodiments of the invention, a damaged peripheral nerve cell.

FIG. 2A illustrates, in accordance with various embodiments of the invention, healthy nerves and blood vessels.

FIG. 2B illustrates, in accordance with various embodiments of the invention, nerves and blood vessels damaged by diabetic peripheral neuropathy (DPN).

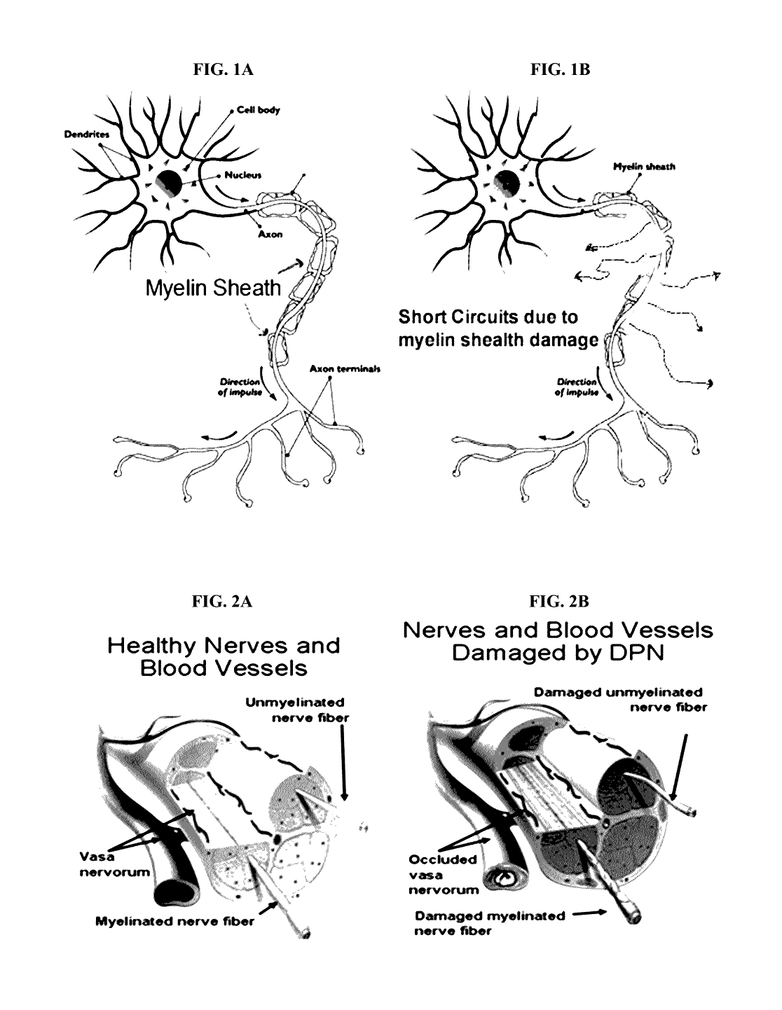

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of neuropathy, delay or slowing of neuropathy, and amelioration or palliation of symptoms associated with neuropathy.

“Diseases,” “conditions” and “disease conditions,” as used herein may include, but are in no way limited to diseases or conditions associated with inflammation. Diseases associated with inflammation may include, but are in no way limited to, any form of neuropathy or arthritis.

As used herein, the term “administering,” refers to the placement of an agent or a composition as disclosed herein into a subject by a method or route which results in at least partial localization of the agents or composition at a desired site. “Route of administration” may refer to any administration pathway known in the art, including but not limited to oral, topical, aerosol, nasal, via inhalation, anal, intra-anal, peri-anal, transmucosal, transdermal, parenteral, enteral, or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the agent or composition may be in the form of solutions or suspensions for infusion or for injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions or emulsions. When formulated for application as a topical spray, the compositions described herein may include alcohol and other common solvents for dilution as a propellant. A topical spray may be particularly advantageous for subjects having neuropathy who would have difficulty reaching an extremity or other location on the body, because it allows for application at a distance. In an embodiment, agent or composition may be provided in a powder form and mixed with a liquid, such as water, to form a beverage. In accordance with the present invention, “administering” can be self-administering. For example, it is considered as “administering” that a subject consumes a composition as disclosed herein.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., neuropathy) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. For example, a subject can be one who exhibits one or more symptoms for a condition or one or more complications related to the condition or a subject who does not exhibit symptoms. A “subject in need” of diagnosis or treatment for a particular condition can be a subject suspected of having that condition, diagnosed as having that condition, already treated or being treated for that condition, not treated for that condition, or at risk of developing that condition.

The term “statistically significant” or “significantly” refers to statistical evidence that there is a difference. It is defined as the probability of making a decision to reject the null hypothesis when the null hypothesis is actually true. The decision is often made using the p-value.

The term “functional” when used in conjunction with “equivalent”, “analog”, “derivative” or “variant” or “fragment” refers to an entity or molecule which possess a biological activity that is substantially similar to a biological activity of the entity or molecule of which it is an equivalent, analog, derivative, variant or fragment thereof.

The Commiphora Mukul extract referenced herein throughout the application contains Gugulsterone at a concentration of 2-10%, 3-7%, 4-6%, or 5%. Thus, Gugulsterone can be used as a substitute for, or in addition to, Commiphora Mukul extract, in order to arrive at the same overall concentration of Gugulsterone present when Commiphora Mukul extract alone is included in any formulation described herein. The structure for Gugulsterone is known to be as follows

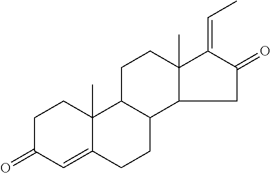 [SMILES: C/C=C1\C(=O)CC2C3CCC4=CC(=O)CCC4(C)C3CCC12C]

Without wishing to be bound by any particular theory, the present invention takes an approach to address chronic diseases by comprehensive care and homeostasis and by addressing various abnormalities like oxygen need, oxidative stress, nutritional deficiencies, and detoxification of accumulated metabolic byproducts.

Various PN treatments as described herein help relieve pain and burning sensation, pins and needles feeling, tingling, numbness and swelling, and fatigue by increasing energy production, and support increased metabolism for nerve cell health, increased blood flow and toxin removal, homeostasis and balance, and strengthening nerves and nerve linings. These PN treatments are safe, have no known side effects, and repair and rejuvenate damaged nerves for longer relief. Additionally, treatments and compositions for arthritis are described herein.

In various embodiments, the compositions may contain ingredients including but limited to one or more or all of vitamins, alpha lipoic acid, taurine, curcumin, piperine, turmeric, natural detox ingredients, and blood flow and cardiovascular support ingredients. Vitamins include but are not limited to B vitamins B2, B3, B6, B12, Folic Acid and B1 in the water insoluble form Benfotiamine for longer availability to facilitate nerve repair; and Vitamin C, which is a biological antioxidant. Alpha lipoic acid is essential for the aerobic metabolism that generates most of the energy for cell function and mobility. It is also a natural biological antioxidant. Taurine is widely distributed in animal tissues. It plays a vital role in membrane stabilization, osmoregulation, calcium signaling and as an antioxidant in reduction of oxidative stress. It is essential for nervous system and cardiovascular function. Taurine can influence (and possibly reverse) defects in nerves, blood flow and motor nerve conduction velocity. Curcumin is one of the most potent antioxidants with clinically proven anti-inflammatory properties, as it downregulates TNF alpha. Piperine and turmeric are natural adjuncts for curcumin activity. Natural detox ingredients for liver, kidney and lymph system include but are not limited to Embellica officinalis, Terminalia ballerica, and Terminalia chebula. Blood flow and cardiovascular support ingredients may include but are not limited to Vinpocetine for nerve blood supply, Commiphora mukul, and Withenia somnifera. Vinpocetine is also a potent antioxidant. As oxidative Stress is the leading cause of inflammation and nerve damage, these PN treatments contain some of the strongest antioxidants found in nature for decreasing oxidative stress.

### Compositions of the Invention

In various embodiments, the present invention provides a composition for treating neuropathy and/or a neuropathy-related condition and/or arthritis. Ingredients of the composition may include but are not limited to the following categories: Taurine, Alpha Lipoic Acid, Phospholipids, Withania somnifera, Triphala Extract, Turmeric, Curcumin, Commiphora Mukul Extract, Piperine, Vinpocetine, and Vitamins, and their functional equivalents, analogs, derivatives, and variants.

In various embodiments, the composition consists of or consists essentially of or comprises 1, 2, 3, 4, 5, 6, 7, 8, 9, or 10 ingredient categories selected from: Taurine, Alpha Lipoic Acid, Phospholipids, Withania somnifera, Triphala Extract, Turmeric, Curcumin, Commiphora Mukul Extract, Piperine, Vinpocetine, and Vitamins, and their functional equivalents, analogs, derivatives, and variants. In various embodiments, the composition consists of or consists essentially of or comprises at least 1, 2, 3, 4, 5, 6, 7, 8, 9, or 10 ingredient categories selected from: Taurine, Alpha Lipoic Acid, Phospholipids, Withania somnifera, Triphala Extract, Turmeric, Curcumin, Commiphora Mukul Extract, Piperine, Vinpocetine, and Vitamins, and their functional equivalents, analogs, derivatives, and variants. In some embodiments, the composition consists of or consists essentially of or comprises at least Taurine and/or Vinpocetine. In some embodiments, the composition further comprises curcumin. In some embodiments, the composition consists of or consists essentially of or comprises at least Taurine. In some embodiments, the composition consists of or consists essentially of or comprises at least Vinpocetine. In some embodiments, the composition consists of or consists essentially of or comprises at least Taurine and Vinpocetine. In various embodiments, the composition consists of or consists essentially of or comprises all ingredient categories of: Taurine, Alpha Lipoic Acid, Phospholipids, Withania somnifera, Triphala Extract, Turmeric, Curcumin, Commiphora Mukul Extract, Piperine, Vinpocetine, and Vitamins, and their functional equivalents, analogs, derivatives, and variants. In some embodiments the composition comprises, consists of, or consists essentially of Withania somnifera and Taurine. In some embodiments, the composition is intended for oral administration.

In various embodiments, the present invention provides a composition for treating neuropathy and/or a neuropathy-related condition and/or arthritis. In various embodiments, the composition consists of or consists essentially of or comprises Taurine and/or Vinpocetine. In some embodiments, the composition further comprises curcumin. In some embodiments, the composition consists of or consists essentially of or comprises Taurine and Vinpocetine. In some embodiments, the composition consists of or consists essentially of or comprises Taurine but not Vinpocetine. In some embodiments, the composition consists of or consists essentially of or comprises Vinpocetine but not Taurine. In various embodiments, the composition further comprises 1, 2, 3, 4, 5, 6, 7, 8, or 9 ingredients of: Alpha Lipoic Acid, Phospholipids, Withania somnifera, Triphala Extract, Turmeric, Curcumin, Commiphora Mukul Extract, Piperine, and Vitamins, and their functional equivalents, analogs, derivatives, and variants. In some embodiments, the composition further comprises Alpha Lipoic Acid. In some embodiments, the composition further comprises Phospholipids (e.g., Phosphatidylcholine (lecithin, PC)). In some embodiments, the composition further comprises Withania somnifera. In some embodiments, the composition further comprises Triphala Extract. In some embodiments, the composition further comprises Turmeric. In some embodiments, the composition further comprises Curcumin. In some embodiments, the composition further comprises Commiphora Mukul Extract. In some embodiments, the composition further comprises Piperine. In some embodiments, the composition further comprises Vitamins. In some embodiments, the composition is intended for oral administration.

Examples of the Phospholipids include but are not limited to Glycerophospholipid, Phosphatidylcholine (lecithin, PC), Phosphatidic acid (phosphatidate, PA), Phosphatidylethanolamine (cephalin, PE), Phosphatidylserine (PS), Phosphoinositides (e.g., Phosphatidylinositol (PI), Phosphatidylinositol phosphate (PIP), Phosphatidylinositol bisphosphate (PIP2) and Phosphatidylinositol triphosphate (PIP3)), Phosphosphingolipids, Ceramide phosphorylcholine (Sphingomyelin, SPH), Ceramide phosphorylethanolamine (Sphingomyelin, Cer-PE) and Ceramide phosphoryllipid. In various embodiments, the Phospholipids contained in the composition are PC, PA, PE, or PS, or a combination thereof. In some embodiments, the Phospholipids contained in the composition are PC. In some embodiments, the composition consists of or consists essentially of or comprises Phosphatidylcholine (lecithin, PC). In some embodiments, the Phosphatidylcholine is Phosphatidylcholine 50% in PG.

Examples of the vitamins include but are not limited to Vitamin C, Vitamin E, Vitamin E Acetate, Vitamin B1, Benfotiamine, Vitamin B2, Vitamin B3, Vitamin B6, Methylcobalamin B12, and Folic Acid. In various embodiments, the Vitamins contained in the composition comprise one or more or all of Vitamin C, Vitamin B1, Benfotiamine, Vitamin B2, Vitamin B3, Vitamin B6, Methylcobalamin B12, and Folic Acid. In various embodiments, the Vitamins contained in the composition comprise Vitamin E Acetate.

In some embodiments, the Withania somnifera is in the form of Ashwagandha, 7.0% withanalides. In some embodiments, the Triphala Extract is in the form of an extract of 1:1:1 ratio of three natural actives: Emblica officinalis, Terminalia bellerica, and Terminalia chebula. In some embodiments, the Curcumin is in the form of Curcumin, 95%. In some embodiments, the Commiphora Mukul Extract is in the form of Commiphora Mukul Extract, 5.0%.

In various embodiments, the composition consists of or consists essentially of or comprises 1, 2, 3, 4, 5, 6, 7, 8, 9, 10, 11, 12, 13, 14, 15, or 16 ingredients selected from: Taurine, Alpha Lipoic Acid, Phospholipids (e.g., PC, PA, PE, and/or PS), Withania somnifera, Triphala Extract, Benfotiamine, Vitamin C, Turmeric, Curcumin, Commiphora Mukul Extract, Vitamin B3, Piperine, Vinpocetine, Vitamin B2, Vitamin B6, Methylcobalamin B12, and Folic Acid. In various embodiments, the composition consists of or consists essentially of or comprises at least 1, 2, 3, 4, 5, 6, 7, 8, 9, 10, 11, 12, 13, 14, 15, or 16 ingredients selected from: Taurine, Alpha Lipoic Acid, Phospholipids (e.g., PC, PA, PE, and/or PS), Withania somnifera, Triphala Extract, Benfotiamine, Vitamin C, Turmeric, Curcumin, Commiphora Mukul Extract, Vitamin B3, Piperine, Vinpocetine, Vitamin B2, Vitamin B6, Methylcobalamin B12, and Folic Acid. In various embodiments, the composition consists of or consists essentially of or comprises all ingredients of: Taurine, Alpha Lipoic Acid, Phospholipids (e.g., PC, PA, PE, and/or PS), Withania somnifera, Triphala Extract, Benfotiamine, Vitamin C, Turmeric, Curcumin, Commiphora Mukul Extract, Vitamin B3, Piperine, Vinpocetine, Vitamin B2, Vitamin B6, Methylcobalamin B12, and Folic Acid. In some embodiments, the composition is intended for oral administration.

As used herein below, the term “% by weight or volume” is meant to indicate the percentage of the overall composition's weight, if the composition is formulated as a solid, and the percentage of the overall composition's volume, if the composition is formulated as a liquid.

In various embodiments, the composition comprises or consists essentially of or consists of Taurine at about 7.70-43.0%, 24.8-26.1%, 26.1-27.3%, 27.3-28.6%, 28.6-29.8%, 29.8-31.0%, 31.0-32.3%, 32.3-33.5%, 33.5-34.8%, 34.8-36.0%, or 36.0-37.3%, or about 31.0% by weight or volume. In various embodiments, the composition comprises or consists essentially of or consists of Alpha Lipoic Acid at about 5.50-32.0%, 12.4-13.0%, 13.0-13.7%, 13.7-14.3%, 14.3-14.9%, 14.9-15.5%, 15.5-16.1%, 16.1-16.8%, 16.8-17.4%, 17.4-18.0%, or 18.0-18.6%, or about 15.5% by weight or volume. In various embodiments, the composition comprises or consists essentially of or consists of Phospholipids (e.g., PC, PA, PE, and/or PS) at about 3.8-39%, 9.3-9.8%, 9.8-10.2%, 10.2-10.7%, 10.7-11.2%, 11.2-11.6%, 11.6-12.1%, 12.1-12.6%, 12.6-13.0%, 13.0-13.5%, or 13.5-14.0%, or about 11.6% by weight or volume. In various embodiments, the composition comprises or consists essentially of or consists of Withania somnifera at about 1.90-19.4%, 7.76-8.15%, 8.15-8.54%, 8.54-8.93%, 8.93-9.31%, 9.31-9.70%, 9.70-10.09%, 10.09-10.48%, 10.48-10.87%, 10.87-11.26%, or 11.26-11.64%, or about 9.70% by weight or volume. In various embodiments, the composition comprises or consists essentially of or consists of Triphala Extract at about 3.80-19.4%, 6.21-6.52%, 6.52-6.83%, 6.83-7.14%, 7.14-7.45%, 7.45-7.76%, 7.76-8.07%, 8.07-8.38%, 8.38-8.69%, 8.69-9.00%, or 9.00-9.31%, or about 7.76% by weight or volume. In various embodiments, the composition comprises or consists essentially of or consists of Benfotiamine at about 1.90-24.0%, 4.66-4.89%, 4.89-5.12%, 5.12-5.36%, 5.36-5.59%, 5.59-5.82%, 5.82-6.05%, 6.05-6.29%, 6.29-6.52%, 6.52-6.75%, or 6.75-6.99%, or about 5.82% by weight or volume. In various embodiments, the composition comprises or consists essentially of or consists of Vitamin C at about 1.90-19.4%, 3.10-3.26%, 3.26-3.42%, 3.42-3.57%, 3.57-3.73%, 3.73-3.88%, 3.88-4.04%, 4.04-4.19%, 4.19-4.35%, 4.35-4.50%, or 4.50-4.66%, or about 3.88% by weight or volume. In various embodiments, the composition comprises or consists essentially of or consists of Turmeric at about 0.90-11.7%, 3.10-3.26%, 3.26-3.42%, 3.42-3.57%, 3.57-3.73%, 3.73-3.88%, 3.88-4.04%, 4.04-4.19%, 4.19-4.35%, 4.35-4.50%, or 4.50-4.66%, or about 3.88% by weight or volume. In various embodiments, the composition comprises or consists essentially of or consists of Curcumin at about 0.90-11.7%, 3.10-3.26%, 3.26-3.42%, 3.42-3.57%, 3.57-3.73%, 3.73-3.88%, 3.88-4.04%, 4.04-4.19%, 4.19-4.35%, 4.35-4.50%, or 4.50-4.66%, or about 3.88% by weight or volume. In various embodiments, the composition comprises or consists essentially of or consists of Commiphora Mukul Extract at about 0.90-9.70%, 3.10-3.26%, 3.26-3.42%, 3.42-3.57%, 3.57-3.73%, 3.73-3.88%, 3.88-4.04%, 4.04-4.19%, 4.19-4.35%, 4.35-4.50%, or 4.50-4.66%, or about 3.88% by weight or volume. In various embodiments, the composition comprises or consists essentially of or consists of Vitamin B3 at about 0.70-3.90%, 1.55-1.63%, 1.63-1.71%, 1.71-1.79%, 1.79-1.86%, 1.86-1.94%, 1.94-2.02%, 2.02-2.10%, 2.10-2.17%, 2.17-2.25%, or 2.25-2.33%, or about 1.94% by weight or volume. In various embodiments, the composition comprises or consists essentially of or consists of Piperine at about 0.03-0.60%, 0.310-0.326%, 0.326-0.342%, 0.342-0.357%, 0.357-0.373%, 0.373-0.388%, 0.388-0.404%, 0.404-0.419%, 0.419-0.435%, 0.435-0.450%, or 0.450-0.466%, or about 0.388% by weight or volume. In various embodiments, the composition comprises or consists essentially of or consists of Vinpocetine at about 0.03-1.60%, 0.186-0.196%, 0.196-0.205%, 0.205-0.214%, 0.214-0.224%, 0.224-0.233%, 0.233-0.242%, 0.242-0.252%, 0.252-0.261%, 0.261-0.270%, or 0.270-0.279%, or about 0.233% by weight or volume. In various embodiments, the composition comprises or consists essentially of or consists of Vitamin B2 at about 0.030-0.400%, 0.124-0.130%, 0.130-0.137%, 0.137-0.143%, 0.143-0.149%, 0.149-0.155%, 0.155-0.161%, 0.161-0.168%, 0.168-0.174%, 0.174-0.180%, or 0.180-0.186%, or about 0.155% by weight or volume. In various embodiments, the composition comprises or consists essentially of or consists of Vitamin B6 at about 0.03-0.40%, 0.124-0.130%, 0.130-0.137%, 0.137-0.143%, 0.143-0.149%, 0.149-0.155%, 0.155-0.161%, 0.161-0.168%, 0.168-0.174%, 0.174-0.180%, or 0.180-0.186%, or about 0.155% by weight or volume. In various embodiments, the composition comprises or consists essentially of or consists of Methylcobalamin B12 at about 0.008-0.120%, 0.0621-0.0652%, 0.0652-0.0683%, 0.0683-0.0714%, 0.0714-0.0745%, 0.0745-0.0776%, 0.0776-0.0807%, 0.0807-0.0838%, 0.0838-0.0869%, 0.0869-0.0900%, or 0.0900-0.0931%, or about 0.0776% by weight or volume. In various embodiments, the composition comprises or consists essentially of or consists of Folic Acid at about 0.004-0.080%, 0.0155-0.0163%, 0.0163-0.0171%, 0.0171-0.0179%, 0.0179-0.0186%, 0.0186-0.0194%, 0.0194-0.0202%, 0.0202-0.0210%, 0.0210-0.0217%, 0.0217-0.0225%, or 0.0225-0.0233%, or about 0.0194% by weight or volume. In some embodiments, the composition comprises or consists essentially of or consists of 1, 2, 3, 4, 5, 6, 7, 8, 9, 10, 11, 12, 13, 14, 15, or 16 of these listed ingredients. In some embodiments, the composition comprises or consists essentially of or consists of at least 1, 2, 3, 4, 5, 6, 7, 8, 9, 10, 11, 12, 13, 14, 15, or 16 of these listed ingredients. In some embodiments, the composition comprises or consists essentially of or consists of all of these listed ingredients. In some embodiments, the composition is intended for oral administration.

In various embodiments, the composition comprises or consists essentially of or consists of at least 1, 2, 3, 4, 5, 6, 7, 8, 9, 10, 11, 12, 13, 14, 15, or 16, or all ingredients of: Taurine at about 7.70-43.0%, 24.8-26.1%, 26.1-27.3%, 27.3-28.6%, 28.6-29.8%, 29.8-31.0%, 31.0-32.3%, 32.3-33.5%, 33.5-34.8%, 34.8-36.0%, or 36.0-37.3%, or about 31.0% by weight or volume; Alpha Lipoic Acid at about 5.5-32.0%, 12.4-13.0%, 13.0-13.7%, 13.7-14.3%, 14.3-14.9%, 14.9-15.5%, 15.5-16.1%, 16.1-16.8%, 16.8-17.4%, 17.4-18.0%, or 18.0-18.6%, or about 15.5% by weight or volume; Phospholipids (e.g., PC, PA, PE, and/or PS) at about 3.8-39%, 9.3-9.8%, 9.8-10.2%, 10.2-10.7%, 10.7-11.2%, 11.2-11.6%, 11.6-12.1%, 12.1-12.6%, 12.6-13.0%, 13.0-13.5%, or 13.5-14.0%, or about 11.6% by weight or volume; Withania somnifera at about 1.90-19.4%, 7.76-8.15%, 8.15-8.54%, 8.54-8.93%, 8.93-9.31%, 9.31-9.70%, 9.70-10.09%, 10.09-10.48%, 10.48-10.87%, 10.87-11.26%, or 11.26-11.64%, or about 9.70% by weight or volume; Triphala Extract at about 3.80-19.4%, 6.21-6.52%, 6.52-6.83%, 6.83-7.14%, 7.14-7.45%, 7.45-7.76%, 7.76-8.07%, 8.07-8.38%, 8.38-8.69%, 8.69-9.00%, or 9.00-9.31%, or about 7.76% by weight or volume; Benfotiamine at about 1.90-24.0%, 4.66-4.89%, 4.89-5.12%, 5.12-5.36%, 5.36-5.59%, 5.59-5.82%, 5.82-6.05%, 6.05-6.29%, 6.29-6.52%, 6.52-6.75%, or 6.75-6.99%, or about 5.82% by weight or volume; Vitamin C at about 1.90-19.4%, 3.10-3.26%, 3.26-3.42%, 3.42-3.57%, 3.57-3.73%, 3.73-3.88%, 3.88-4.04%, 4.04-4.19%, 4.19-4.35%, 4.35-4.50%, or 4.50-4.66%, or about 3.88% by weight or volume; Turmeric at about 0.90-11.7%, 3.10-3.26%, 3.26-3.42%, 3.42-3.57%, 3.57-3.73%, 3.73-3.88%, 3.88-4.04%, 4.04-4.19%, 4.19-4.35%, 4.35-4.50%, or 4.50-4.66%, or about 3.88% by weight or volume; Curcumin at about 0.90-11.7%, 3.10-3.26%, 3.26-3.42%, 3.42-3.57%, 3.57-3.73%, 3.73-3.88%, 3.88-4.04%, 4.04-4.19%, 4.19-4.35%, 4.35-4.50%, or 4.50-4.66%, or about 3.88% by weight or volume; Commiphora Mukul Extract at about 0.90-9.70%, 3.10-3.26%, 3.26-3.42%, 3.42-3.57%, 3.57-3.73%, 3.73-3.88%, 3.88-4.04%, 4.04-4.19%, 4.19-4.35%, 4.35-4.50%, or 4.50-4.66%, or about 3.88% by weight or volume; Vitamin B3 at about 0.70-3.90%, 1.55-1.63%, 1.63-1.71%, 1.71-1.79%, 1.79-1.86%, 1.86-1.94%, 1.94-2.02%, 2.02-2.10%, 2.10-2.17%, 2.17-2.25%, or 2.25-2.33%, or about 1.94% by weight or volume; Piperine at about 0.03-0.60%, 0.310-0.326%, 0.326-0.342%, 0.342-0.357%, 0.357-0.373%, 0.373-0.388%, 0.388-0.404%, 0.404-0.419%, 0.419-0.435%, 0.435-0.450%, or 0.450-0.466%, or about 0.388% by weight or volume; Vinpocetine at about 0.03-1.60%, 0.186-0.196%, 0.196-0.205%, 0.205-0.214%, 0.214-0.224%, 0.224-0.233%, 0.233-0.242%, 0.242-0.252%, 0.252-0.261%, 0.261-0.270%, or 0.270-0.279%, or about 0.233% by weight or volume; Vitamin B2 at about 0.030-0.400%, 0.124-0.130%, 0.130-0.137%, 0.137-0.143%, 0.143-0.149%, 0.149-0.155%, 0.155-0.161%, 0.161-0.168%, 0.168-0.174%, 0.174-0.180%, or 0.180-0.186%, or about 0.155% by weight or volume; Vitamin B6 at about 0.03-0.40%, 0.124-0.130%, 0.130-0.137%, 0.137-0.143%, 0.143-0.149%, 0.149-0.155%, 0.155-0.161%, 0.161-0.168%, 0.168-0.174%, 0.174-0.180%, or 0.180-0.186%, or about 0.155% by weight or volume; Methylcobalamin B12 at about 0.008-0.120%, 0.0621-0.0652%, 0.0652-0.0683%, 0.0683-0.0714%, 0.0714-0.0745%, 0.0745-0.0776%, 0.0776-0.0807%, 0.0807-0.0838%, 0.0838-0.0869%, 0.0869-0.0900%, or 0.0900-0.0931%, or about 0.0776% by weight or volume; and Folic Acid at about 0.004-0.080%, 0.0155-0.0163%, 0.0163-0.0171%, 0.0171-0.0179%, 0.0179-0.0186%, 0.0186-0.0194%, 0.0194-0.0202%, 0.0202-0.0210%, 0.0210-0.0217%, 0.0217-0.0225%, or 0.0225-0.0233%, or about 0.0194% by weight or volume. In some embodiments, the composition is intended for oral administration.

In various embodiments, the composition comprises or consists essentially of or consists of Taurine at about 50-275, 160-168, 168-176, 176-184, 184-192, 192-200, 200-208, 208-216, 216-224, 224-232, or 232-240 mg, or about 200 mg. In various embodiments, the composition comprises or consists essentially of or consists of Alpha Lipoic Acid at about 37.5-200, 80-84, 84-88, 88-92, 92-96, 96-100, 100-104, 104-108, 108-112, 112-116, or 116-120 mg, or about 100 mg. In various embodiments, the composition comprises or consists essentially of or consists of Phospholipids (e.g., PC, PA, PE, and/or PS) at about 25.0-250, 60.0-63.0, 63.0-66.0, 66.0-69.0, 69.0-72.0, 72.0-75.0, 75.0-78.0, 78.0-81.0, 81.0-84.0, 84.0-87.0, or 87.0-90.0 mg, or about 75.0 mg. In various embodiments, the composition comprises or consists essentially of or consists of Withania somnifera at about 12.5-125, 50.0-52.5, 52.5-55.0, 55.0-57.5, 57.5-60.0, 60.0-62.5, 62.5-65.0, 65.0-67.5, 67.5-70.0, 70.0-72.5, or 72.5-75.0 mg, or about 62.5 mg. In various embodiments, the composition comprises or consists essentially of or consists of Triphala Extract at about 25.0-125, 40.0-42.0, 42.0-44.0, 44.0-46.0, 46.0-48.0, 48.0-50.0, 50.0-52.0, 52.0-54.0, 54.0-56.0, 56.0-58.0, or 58.0-60.0 mg, or about 50.0 mg. In various embodiments, the composition comprises or consists essentially of or consists of Benfotiamine at about 12.5-150, 30.0-31.5, 31.5-33.0, 33.0-34.5, 34.5-36.0, 36.0-37.5, 37.5-39.0, 39.0-40.5, 40.5-42.0, 42.0-43.5, or 43.5-45.0 mg, or about 37.5 mg. In various embodiments, the composition comprises or consists essentially of or consists of Vitamin C at about 12.5-125, 20.0-21.0, 21.0-22.0, 22.0-23.0, 23.0-24.0, 24.0-25.0, 25.0-26.0, 26.0-27.0, 27.0-28.0, 28.0-29.0, or 29.0-30.0 mg, or about 25.0 mg. In various embodiments, the composition comprises or consists essentially of or consists of Turmeric at about 6.25-75.0, 20.0-21.0, 21.0-22.0, 22.0-23.0, 23.0-24.0, 24.0-25.0, 25.0-26.0, 26.0-27.0, 27.0-28.0, 28.0-29.0, or 29.0-30.0 mg, or about 25.0 mg. In various embodiments, the composition comprises or consists essentially of or consists of Curcumin at about 6.25-75.0, 20.0-21.0, 21.0-22.0, 22.0-23.0, 23.0-24.0, 24.0-25.0, 25.0-26.0, 26.0-27.0, 27.0-28.0, 28.0-29.0, or 29.0-30.0 mg, or about 25.0 mg. In various embodiments, the composition comprises or consists essentially of or consists of Commiphora Mukul Extract at about 6.25-62.5, 20.0-21.0, 21.0-22.0, 22.0-23.0, 23.0-24.0, 24.0-25.0, 25.0-26.0, 26.0-27.0, 27.0-28.0, 28.0-29.0, or 29.0-30.0 mg, or about 25.0 mg. In various embodiments, the composition comprises or consists essentially of or consists of Vitamin B3 at about 5.0-25.0, 10.0-10.5, 10.5-11.0, 11.0-11.5, 11.5-12.0, 12.0-12.5, 12.5-13.0, 13.0-13.5, 13.5-14.0, 14.0-14.5, or 14.5-15.0 mg, or about 12.5 mg. In various embodiments, the composition comprises or consists essentially of or consists of Piperine at about 0.25-3.75, 2.00-2.10, 2.10-2.20, 2.20-2.30, 2.30-2.40, 2.40-2.50, 2.50-2.60, 2.60-2.70, 2.70-2.80, 2.80-2.90, or 2.90-3.00 mg, or about 2.50 mg. In various embodiments, the composition comprises or consists essentially of or consists of Vinpocetine at about 0.25-10.0, 1.20-1.26, 1.26-1.32, 1.32-1.38, 1.38-1.44, 1.44-1.50, 1.50-1.56, 1.56-1.62, 1.62-1.68, 1.68-1.74, or 1.74-1.80 mg, or about 1.50 mg. In various embodiments, the composition comprises or consists essentially of or consists of Vitamin B2 at about 0.25-2.50, 0.80-0.84, 0.84-0.88, 0.88-0.92, 0.92-0.96, 0.96-1.00, 1.00-1.04, 1.04-1.08, 1.08-1.12, 1.12-1.16, or 1.16-1.20 mg, or about 1.00 mg. In various embodiments, the composition comprises or consists essentially of or consists of Vitamin B6 at about 0.25-2.50, 0.80-0.84, 0.84-0.88, 0.88-0.92, 0.92-0.96, 0.96-1.00, 1.00-1.04, 1.04-1.08, 1.08-1.12, 1.12-1.16, or 1.16-1.20 mg, or about 1.00 mg. In various embodiments, the composition comprises or consists essentially of or consists of Methylcobalamin B12 at about 0.050-0.750, 0.400-0.420, 0.420-0.440, 0.440-0.460, 0.460-0.480, 0.480-0.500, 0.500-0.520, 0.520-0.540, 0.540-0.560, 0.560-0.580, or 0.580-0.600 mg, or about 0.500 mg. In various embodiments, the composition comprises or consists essentially of or consists of Folic Acid at about 0.025-0.500, 0.100-0.105, 0.105-0.110, 0.110-0.115, 0.115-0.120, 0.120-0.125, 0.125-0.130, 0.130-0.135, 0.135-0.140, 0.140-0.145, or 0.145-0.150 mg, or about 0.125 mg. In some embodiments, the composition comprises or consists essentially of or consists of 1, 2, 3, 4, 5, 6, 7, 8, 9, 10, 11, 12, 13, 14, 15, or 16 of these listed ingredients. In some embodiments, the composition comprises or consists essentially of or consists of at least 1, 2, 3, 4, 5, 6, 7, 8, 9, 10, 11, 12, 13, 14, 15, or 16 of these listed ingredients. In some embodiments, the composition comprises or consists essentially of or consists of all of these listed ingredients. In some embodiments, the composition is intended for oral administration. In various embodiments, a capsule or tablet of the oral formulation comprises about 400-450, 450-500, 500-550, 550-600, 600-650, 650-700, 700-750, 750-800, 800-850, or 850-900 mg of a composition as disclosed herein.

In various embodiments, the composition comprises or consists essentially of or consists of at least 1, 2, 3, 4, 5, 6, 7, 8, 9, 10, 11, 12, 13, 14, 15, or 16, or all ingredients of: Taurine at about 50-275, 160-168, 168-176, 176-184, 184-192, 192-200, 200-208, 208-216, 216-224, 224-232, or 232-240 mg, or about 200 mg; Alpha Lipoic Acid at about 37.5-200, 80-84, 84-88, 88-92, 92-96, 96-100, 100-104, 104-108, 108-112, 112-116, or 116-120 mg, or about 100 mg; Phospholipids (e.g., PC, PA, PE, and/or PS) at about 25.0-250, 60.0-63.0, 63.0-66.0, 66.0-69.0, 69.0-72.0, 72.0-75.0, 75.0-78.0, 78.0-81.0, 81.0-84.0, 84.0-87.0, or 87.0-90.0 mg, or about 75.0 mg; Withania somnifera at about 12.5-125, 50.0-52.5, 52.5-55.0, 55.0-57.5, 57.5-60.0, 60.0-62.5, 62.5-65.0, 65.0-67.5, 67.5-70.0, 70.0-72.5, or 72.5-75.0 mg, or about 62.5 mg; Triphala Extract at about 25.0-125, 40.0-42.0, 42.0-44.0, 44.0-46.0, 46.0-48.0, 48.0-50.0, 50.0-52.0, 52.0-54.0, 54.0-56.0, 56.0-58.0, or 58.0-60.0 mg, or about 50.0 mg; Benfotiamine at about 12.5-150, 30.0-31.5, 31.5-33.0, 33.0-34.5, 34.5-36.0, 36.0-37.5, 37.5-39.0, 39.0-40.5, 40.5-42.0, 42.0-43.5, or 43.5-45.0 mg, or about 37.5 mg; Vitamin C at about 12.5-125, 20.0-21.0, 21.0-22.0, 22.0-23.0, 23.0-24.0, 24.0-25.0, 25.0-26.0, 26.0-27.0, 27.0-28.0, 28.0-29.0, or 29.0-30.0 mg, or about 25.0 mg; Turmeric at about 6.25-75.0, 20.0-21.0, 21.0-22.0, 22.0-23.0, 23.0-24.0, 24.0-25.0, 25.0-26.0, 26.0-27.0, 27.0-28.0, 28.0-29.0, or 29.0-30.0 mg, or about 25.0 mg; Curcumin at about 6.25-75.0, 20.0-21.0, 21.0-22.0, 22.0-23.0, 23.0-24.0, 24.0-25.0, 25.0-26.0, 26.0-27.0, 27.0-28.0, 28.0-29.0, or 29.0-30.0 mg, or about 25.0 mg; Commiphora Mukul Extract at about 6.25-62.5, 20.0-21.0, 21.0-22.0, 22.0-23.0, 23.0-24.0, 24.0-25.0, 25.0-26.0, 26.0-27.0, 27.0-28.0, 28.0-29.0, or 29.0-30.0 mg, or about 25.0 mg; Vitamin B3 at about 5.0-25.0, 10.0-10.5, 10.5-11.0, 11.0-11.5, 11.5-12.0, 12.0-12.5, 12.5-13.0, 13.0-13.5, 13.5-14.0, 14.0-14.5, or 14.5-15.0 mg, or about 12.5 mg; Piperine at about 0.25-3.75, 2.00-2.10, 2.10-2.20, 2.20-2.30, 2.30-2.40, 2.40-2.50, 2.50-2.60, 2.60-2.70, 2.70-2.80, 2.80-2.90, or 2.90-3.00 mg, or about 2.50 mg; Vinpocetine at about 0.25-10.0, 1.20-1.26, 1.26-1.32, 1.32-1.38, 1.38-1.44, 1.44-1.50, 1.50-1.56, 1.56-1.62, 1.62-1.68, 1.68-1.74, or 1.74-1.80 mg, or about 1.50 mg; Vitamin B2 at about 0.25-2.50, 0.80-0.84, 0.84-0.88, 0.88-0.92, 0.92-0.96, 0.96-1.00, 1.00-1.04, 1.04-1.08, 1.08-1.12, 1.12-1.16, or 1.16-1.20 mg, or about 1.00 mg; Vitamin B6 at about 0.25-2.50, 0.80-0.84, 0.84-0.88, 0.88-0.92, 0.92-0.96, 0.96-1.00, 1.00-1.04, 1.04-1.08, 1.08-1.12, 1.12-1.16, or 1.16-1.20 mg, or about 1.00 mg; Methylcobalamin B12 at about 0.050-0.750, 0.400-0.420, 0.420-0.440, 0.440-0.460, 0.460-0.480, 0.480-0.500, 0.500-0.520, 0.520-0.540, 0.540-0.560, 0.560-0.580, or 0.580-0.600 mg, or about 0.500 mg; and Folic Acid at about 0.025-0.500, 0.100-0.105, 0.105-0.110, 0.110-0.115, 0.115-0.120, 0.120-0.125, 0.125-0.130, 0.130-0.135, 0.135-0.140, 0.140-0.145, or 0.145-0.150 mg, or about 0.125 mg. In some embodiments, the composition is intended for oral administration. In various embodiments, a capsule or tablet of the oral formulation comprises about 400-450, 450-500, 500-550, 550-600, 600-650, 650-700, 700-750, 750-800, 800-850, or 850-900 mg of a composition as disclosed herein.

In various embodiments, the composition consists of or consists essentially of or comprises 1, 2, 3, or 4 ingredients selected from: Phospholipids (e.g., PC, PA, PE, and/or PS), Boswellia Acetyl-11-keto-β-boswellic acid (AKBA), Vitamins, Lipoic Acid, and Vinpocetine. Boswellia AKBA as used herein throughout the application refers to Boswellia extract which contains AKBA at a concentration ranging from 5-50%, 10-40%, 15-30%, or 20-25%. The structure of Keto Boswellic acid is as follows:

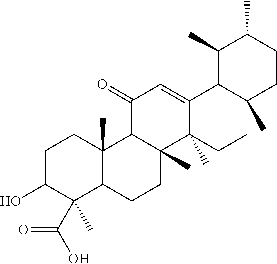 [SMILES: CC[C@]1(C)C(C2[C@H](C)CC[C@@H](C)[C@@H]2C)=CC(=O)C2[C@@]3(C)CCC(O)[C@](C)(C(=O)O)C3CC[C@]21C]

In AKBA the OH group at the alpha position is acetylated.

In various embodiments, the composition consists of or consists essentially of or comprises at least 1, 2, 3, or 4 ingredients selected from: Phospholipids (e.g., PC, PA, PE, and/or PS), Boswellia AKBA, Vitamins, Lipoic Acid, and Vinpocetine. In some embodiments, the composition consists of or consists essentially of or comprises at least Boswellia AKBA and Vinpocentin. In some embodiments, the composition further comprises curcumin. In various embodiments, the composition consists of or consists essentially of or comprises all ingredients of: Phospholipids (e.g., PC, PA, PE, and/or PS), Boswellia AKBA, Vitamins, Lipoic Acid, and Vinpocetine. In various embodiments, the Vitamins comprise Vitamin E and/or Vitamin E Acetate. In various embodiments, the composition further comprises 1, 2, 3, 4, 5, 6, 7, 8, 9, 10, 11, 12, 13, 14, 15, 16, 17, 18, 19, 20, 21, 22, or 23 ingredients of: Water, Sesame oil, Cetearyl Alc. & Cetearyl Gluco., Cetearyl Alcohol, Glyceryl & PEG-100 Stearate, Menthol, Glycerin Monolaurate, Propylene Glycol, Glycerin, Cetyl Myristoleate, Cetyl alcohol, Dimethicone, Shea Butter, Winter green oil, Aloe 10X, Polyhexameth. Biguanide, Cyclopentasiloxane, Ascorbyl Palmitate, Ethyl Lauryl Arginate, Ultrez 20 Carbomer, BHA, Dehydro Acetic Acid, and Sesamin Complex. In some embodiments, the compositions described above and below are formulated as topical formulations. In some embodiments, the compositions described above and below are formulated as oral formulations.

In various embodiments, the composition comprises or consists essentially of or consists of at least 1, 2, 3, or 4, or all ingredients of: Phospholipids (e.g., PC, PA, PE, and/or PS) at about 2.40-2.52%, 2.52-2.64%, 2.64-2.76%, 2.76-2.88%, 2.88-3.00%, 3.00-3.12%, 3.12-3.24%, 3.24-3.36%, 3.36-3.48%, or 3.48-3.60%, or about 3.00% by weight or volume; Boswellia AKBA at about 1.60-1.68%, 1.68-1.76%, 1.76-1.84%, 1.84-1.92%, 1.92-2.00%, 2.00-2.08%, 2.08-2.16%, 2.16-2.24%, 2.24-2.32%, or 2.32-2.40%, or about 2.00% by weight or volume; Vitamin E Acetate at about 0.80-0.84%, 0.84-0.88%, 0.88-0.92%, 0.92-0.96%, 0.96-1.00%, 1.00-1.04%, 1.04-1.08%, 1.08-1.12%, 1.12-1.16%, or 1.16-1.20%, or about 1.00% by weight or volume; Lipoic Acid at about 0.80-0.84%, 0.84-0.88%, 0.88-0.92%, 0.92-0.96%, 0.96-1.00%, 1.00-1.04%, 1.04-1.08%, 1.08-1.12%, 1.12-1.16%, or 1.16-1.20%, or about 1.00% by weight or volume; and Vinpocetine at about 0.080-0.084%, 0.084-0.088%, 0.088-0.092%, 0.092-0.096%, 0.096-0.100%, 0.100-0.104%, 0.104-0.108%, 0.108-0.112%, 0.112-0.116%, or 0.116-0.120%, or about 0.100% by weight or volume. In some embodiments, the composition is intended for topical administration.

In various embodiments, the composition comprises or consists essentially of or consists of at least 1, 2, 3, or 4, or all ingredients of: Phospholipids (e.g., PC, PA, PE, and/or PS) at about 96-101, 101-106, 106-110, 110-115, 115-120, 120-125, 125-130, 130-134, 134-139, or 139-144 g, or about 120 g; Boswellia AKBA at about 64.0-67.2, 67.2-70.4, 70.4-73.6, 73.6-76.8, 76.8-80.0, 80.0-83.2, 83.2-86.4, 86.4-89.6, 89.6-92.8, or 92.8-96.0 g, or about 80.0 g; Vitamin E Acetate at about 32.0-33.6, 33.6-35.2, 35.2-36.8, 36.8-38.4, 38.4-40.0, 40.0-41.6, 41.6-43.2, 43.2-44.8, 44.8-46.4, or 46.4-48.0 g, or about 40.0 g; Lipoic Acid at about 32.0-33.6, 33.6-35.2, 35.2-36.8, 36.8-38.4, 38.4-40.0, 40.0-41.6, 41.6-43.2, 43.2-44.8, 44.8-46.4, or 46.4-48.0 g, or about 40.0 g; and Vinpocetine at about 3.20-3.36, 3.36-3.52, 3.52-3.68, 3.68-3.84, 3.84-4.00, 4.00-4.16, 4.16-4.32, 4.32-4.48, 4.48-4.64, or 4.64-4.80 g, or about 4.00 g. In some embodiments, the composition is intended for topical administration. In some embodiments, the total weight of the topical composition is 15-100 g, or 20-80 g, 30-70 g, or 40-60 g.

In various embodiments, the composition consists of or consists essentially of or comprises 1, 2, 3, 4, 5, 6, 7, 8, 9, 10, 11, 12, 13, 14, 15, 16, 17, 18, 19, 20, 21, 22, 23, 24, 25, 26, or 27 ingredients selected from: Water, Sesame oil, Cetearyl Alc. & Cetearyl Gluco., Cetearyl Alcohol, Glyceryl & PEG-100 Stearate, Phospholipids (e.g., PC, PA, PE, and/or PS), Menthol, Glycerin Monolaurate, Boswellia AKBA, Propylene Glycol, Glycerin, Cetyl Myristoleate, Cetyl alcohol, Dimethicone, Shea Butter, Vitamin E Acetate, Lipoic Acid, Winter green oil, Aloe 10X, Polyhexameth. Biguanide, Cyclopentasiloxane, Ascorbyl Palmitate, Ethyl Lauryl Arginate, Ultrez 20 Carbomer, BHA, Dehydro Acetic Acid, Sesamin Complex, and Vinpocetine. In some embodiments, the composition is intended for topical administration.

In various embodiments, the composition consists of or consists essentially of or comprises at least 1, 2, 3, 4, 5, 6, 7, 8, 9, 10, 11, 12, 13, 14, 15, 16, 17, 18, 19, 20, 21, 22, 23, 24, 25, 26, or 27 ingredients selected from: Water, Sesame oil, Cetearyl Alc. & Cetearyl Gluco., Cetearyl Alcohol, Glyceryl & PEG-100 Stearate, Phospholipids (e.g., PC, PA, PE, and/or PS), Menthol, Glycerin Monolaurate, Boswellia AKBA, Propylene Glycol, Glycerin, Cetyl Myristoleate, Cetyl alcohol, Dimethicone, Shea Butter, Vitamin E Acetate, Lipoic Acid, Winter green oil, Aloe 10X, Polyhexameth. Biguanide, Cyclopentasiloxane, Ascorbyl Palmitate, Ethyl Lauryl Arginate, Ultrez 20 Carbomer, BHA, Dehydro Acetic Acid, Sesamin Complex, and Vinpocetine. In some embodiments, the composition is intended for topical administration.

In various embodiments, the composition consists of or consists essentially of or comprises all ingredients of: Water, Sesame oil, Cetearyl Alc. & Cetearyl Gluco., Cetearyl Alcohol, Glyceryl & PEG-100 Stearate, Phospholipids (e.g., PC, PA, PE, and/or PS), Menthol, Glycerin Monolaurate, Boswellia AKBA, Propylene Glycol, Glycerin, Cetyl Myristoleate, Cetyl alcohol, Dimethicone, Shea Butter, Vitamin E Acetate, Lipoic Acid, Winter green oil, Aloe 10X, Polyhexameth. Biguanide, Cyclopentasiloxane, Ascorbyl Palmitate, Ethyl Lauryl Arginate, Ultrez 20 Carbomer, BHA, Dehydro Acetic Acid, Sesamin Complex, and Vinpocetine. In some embodiments, the composition is intended for topical administration.

In some embodiments, the Boswellia AKBA is in the form of Boswellia 30% AKBA. In some embodiments, the Polyhexameth. Biguanide is in the form of Polyhexameth. Biguanide 20%. In some embodiments, the Ethyl Lauryl Arginate is in the form of Ethyl Lauryl Arginate 20%.

In various embodiments, the composition comprises or consists essentially of or consists of Water at about 43.4-45.5%, 45.5-47.7%, 47.7-49.9%, 49.9-52.0%, 52.0-54.2%, 54.2-56.4%, 56.4-58.5%, 58.5-60.7%, 60.7-62.9%, or 62.9-65.0%, or about 54.2% by weight or volume. In various embodiments, the composition comprises or consists essentially of or consists of Sesame oil at about 9.6-10.1%, 10.1-10.6%, 10.6-11.0%, 11.0-11.5%, 11.5-12.0%, 12.0-12.5%, 12.5-13.0%, 13.0-13.4%, 13.4-13.9%, or 13.9-14.4%, or about 12.0% by weight or volume. In various embodiments, the composition comprises or consists essentially of or consists of Cetearyl Alc. & Cetearyl Gluco. at about 2.40-2.52%, 2.52-2.64%, 2.64-2.76%, 2.76-2.88%, 2.88-3.00%, 3.00-3.12%, 3.12-3.24%, 3.24-3.36%, 3.36-3.48%, or 3.48-3.60%, or about 3.00% by weight or volume. In various embodiments, the composition comprises or consists essentially of or consists of Cetearyl Alcohol at about 2.40-2.52%, 2.52-2.64%, 2.64-2.76%, 2.76-2.88%, 2.88-3.00%, 3.00-3.12%, 3.12-3.24%, 3.24-3.36%, 3.36-3.48%, or 3.48-3.60%, or about 3.00% by weight or volume. In various embodiments, the composition comprises or consists essentially of or consists of Glyceryl & PEG-100 Stearate at about 2.40-2.52%, 2.52-2.64%, 2.64-2.76%, 2.76-2.88%, 2.88-3.00%, 3.00-3.12%, 3.12-3.24%, 3.24-3.36%, 3.36-3.48%, or 3.48-3.60%, or about 3.00% by weight or volume. In various embodiments, the composition comprises or consists essentially of or consists of Phospholipids (e.g., PC, PA, PE, and/or PS) at about 2.40-2.52%, 2.52-2.64%, 2.64-2.76%, 2.76-2.88%, 2.88-3.00%, 3.00-3.12%, 3.12-3.24%, 3.24-3.36%, 3.36-3.48%, or 3.48-3.60%, or about 3.00% by weight or volume. In various embodiments, the composition comprises or consists essentially of or consists of Menthol at about 1.68-1.76%, 1.76-1.85%, 1.85-1.93%, 1.93-2.02%, 2.02-2.10%, 2.10-2.18%, 2.18-2.27%, 2.27-2.35%, 2.35-2.44%, or 2.44-2.52%, or about 2.10% by weight or volume. In various embodiments, the composition comprises or consists essentially of or consists of Glycerin Monolaurate at about 1.60-1.68%, 1.68-1.76%, 1.76-1.84%, 1.84-1.92%, 1.92-2.00%, 2.00-2.08%, 2.08-2.16%, 2.16-2.24%, 2.24-2.32%, or 2.32-2.40%, or about 2.00% by weight or volume. In various embodiments, the composition comprises or consists essentially of or consists of Boswellia AKBA at about 1.60-1.68%, 1.68-1.76%, 1.76-1.84%, 1.84-1.92%, 1.92-2.00%, 2.00-2.08%, 2.08-2.16%, 2.16-2.24%, 2.24-2.32%, or 2.32-2.40%, or about 2.00% by weight or volume. In various embodiments, the composition comprises or consists essentially of or consists of Propylene Glycol at about 1.60-1.68%, 1.68-1.76%, 1.76-1.84%, 1.84-1.92%, 1.92-2.00%, 2.00-2.08%, 2.08-2.16%, 2.16-2.24%, 2.24-2.32%, or 2.32-2.40%, or about 2.00% by weight or volume. In various embodiments, the composition comprises or consists essentially of or consists of Glycerin at about 1.60-1.68%, 1.68-1.76%, 1.76-1.84%, 1.84-1.92%, 1.92-2.00%, 2.00-2.08%, 2.08-2.16%, 2.16-2.24%, 2.24-2.32%, or 2.32-2.40%, or about 2.00% by weight or volume. In various embodiments, the composition comprises or consists essentially of or consists of Cetyl Myristoleate at about 0.80-0.84%, 0.84-0.88%, 0.88-0.92%, 0.92-0.96%, 0.96-1.00%, 1.00-1.04%, 1.04-1.08%, 1.08-1.12%, 1.12-1.16%, or 1.16-1.20%, or about 1.00% by weight or volume. In various embodiments, the composition comprises or consists essentially of or consists of Cetyl alcohol at about 0.80-0.84%, 0.84-0.88%, 0.88-0.92%, 0.92-0.96%, 0.96-1.00%, 1.00-1.04%, 1.04-1.08%, 1.08-1.12%, 1.12-1.16%, or 1.16-1.20%, or about 1.00% by weight or volume. In various embodiments, the composition comprises or consists essentially of or consists of Dimethicone at about 0.80-0.84%, 0.84-0.88%, 0.88-0.92%, 0.92-0.96%, 0.96-1.00%, 1.00-1.04%, 1.04-1.08%, 1.08-1.12%, 1.12-1.16%, or 1.16-1.20%, or about 1.00% by weight or volume. In various embodiments, the composition comprises or consists essentially of or consists of Shea Butter at about 0.80-0.84%, 0.84-0.88%, 0.88-0.92%, 0.92-0.96%, 0.96-1.00%, 1.00-1.04%, 1.04-1.08%, 1.08-1.12%, 1.12-1.16%, or 1.16-1.20%, or about 1.00% by weight or volume. In various embodiments, the composition comprises or consists essentially of or consists of Vitamin E Acetate at about 0.80-0.84%, 0.84-0.88%, 0.88-0.92%, 0.92-0.96%, 0.96-1.00%, 1.00-1.04%, 1.04-1.08%, 1.08-1.12%, 1.12-1.16%, or 1.16-1.20%, or about 1.00% by weight or volume. In various embodiments, the composition comprises or consists essentially of or consists of Lipoic Acid at about 0.80-0.84%, 0.84-0.88%, 0.88-0.92%, 0.92-0.96%, 0.96-1.00%, 1.00-1.04%, 1.04-1.08%, 1.08-1.12%, 1.12-1.16%, or 1.16-1.20%, or about 1.00% by weight or volume. In various embodiments, the composition comprises or consists essentially of or consists of Winter green oil at about 0.80-0.84%, 0.84-0.88%, 0.88-0.92%, 0.92-0.96%, 0.96-1.00%, 1.00-1.04%, 1.04-1.08%, 1.08-1.12%, 1.12-1.16%, or 1.16-1.20%, or about 1.00% by weight or volume. In various embodiments, the composition comprises or consists essentially of or consists of Aloe 10X at about 0.80-0.84%, 0.84-0.88%, 0.88-0.92%, 0.92-0.96%, 0.96-1.00%, 1.00-1.04%, 1.04-1.08%, 1.08-1.12%, 1.12-1.16%, or 1.16-1.20%, or about 1.00% by weight or volume. In various embodiments, the composition comprises or consists essentially of or consists of Polyhexameth. Biguanide at about 0.80-0.84%, 0.84-0.88%, 0.88-0.92%, 0.92-0.96%, 0.96-1.00%, 1.00-1.04%, 1.04-1.08%, 1.08-1.12%, 1.12-1.16%, or 1.16-1.20%, or about 1.00% by weight or volume. In various embodiments, the composition comprises or consists essentially of or consists of Cyclopentasiloxane at about 0.640-0.672%, 0.672-0.704%, 0.704-0.736%, 0.736-0.768%, 0.768-0.800%, 0.800-0.832%, 0.832-0.864%, 0.864-0.896%, 0.896-0.928%, or 0.928-0.960%, or about 0.800% by weight or volume. In various embodiments, the composition comprises or consists essentially of or consists of Ascorbyl Palmitate at about 0.400-0.420%, 0.420-0.440%, 0.440-0.460%, 0.460-0.480%, 0.480-0.500%, 0.500-0.520%, 0.520-0.540%, 0.540-0.560%, 0.560-0.580%, or 0.580-0.600%, or about 0.500% by weight or volume. In various embodiments, the composition comprises or consists essentially of or consists of Ethyl Lauryl Arginate at about 0.400-0.420%, 0.420-0.440%, 0.440-0.460%, 0.460-0.480%, 0.480-0.500%, 0.500-0.520%, 0.520-0.540%, 0.540-0.560%, 0.560-0.580%, or 0.580-0.600%, or about 0.500% by weight or volume. In various embodiments, the composition comprises or consists essentially of or consists of Ultrez 20 Carbomer at about 0.240-0.252%, 0.252-0.264%, 0.264-0.276%, 0.276-0.288%, 0.288-0.300%, 0.300-0.312%, 0.312-0.324%, 0.324-0.336%, 0.336-0.348%, or 0.348-0.360%, or about 0.300% by weight or volume. In various embodiments, the composition comprises or consists essentially of or consists of BHA at about 0.160-0.168%, 0.168-0.176%, 0.176-0.184%, 0.184-0.192%, 0.192-0.200%, 0.200-0.208%, 0.208-0.216%, 0.216-0.224%, 0.224-0.232%, or 0.232-0.240%, or about 0.200% by weight or volume. In various embodiments, the composition comprises or consists essentially of or consists of Dehydro Acetic Acid at about 0.160-0.168%, 0.168-0.176%, 0.176-0.184%, 0.184-0.192%, 0.192-0.200%, 0.200-0.208%, 0.208-0.216%, 0.216-0.224%, 0.224-0.232%, or 0.232-0.240%, or about 0.200% by weight or volume. In various embodiments, the composition comprises or consists essentially of or consists of Sesamin Complex at about 0.080-0.084%, 0.084-0.088%, 0.088-0.092%, 0.092-0.096%, 0.096-0.100%, 0.100-0.104%, 0.104-0.108%, 0.108-0.112%, 0.112-0.116%, or 0.116-0.120%, or about 0.100% by weight or volume. In various embodiments, the composition comprises or consists essentially of or consists of Vinpocetine at about 0.080-0.084%, 0.084-0.088%, 0.088-0.092%, 0.092-0.096%, 0.096-0.100%, 0.100-0.104%, 0.104-0.108%, 0.108-0.112%, 0.112-0.116%, or 0.116-0.120%, or about 0.100% by weight or volume. In some embodiments, the composition comprises or consists essentially of or consists of 1, 2, 3, 4, 5, 6, 7, 8, 9, 10, 11, 12, 13, 14, 15, 16, 17, 18, 19, 20, 21, 22, 23, 24, 25, 26, or 27 of these listed ingredients. In some embodiments, the composition comprises or consists essentially of or consists of at least 1, 2, 3, 4, 5, 6, 7, 8, 9, 10, 11, 12, 13, 14, 15, 16, 17, 18, 19, 20, 21, 22, 23, 24, 25, 26, or 27 of these listed ingredients. In some embodiments, the composition comprises or consists essentially of or consists of all of these listed ingredients. In some embodiments, the composition is intended for topical administration.

In various embodiments, the composition comprises or consists essentially of or consists of Water at about 1734-1821, 1821-1908, 1908-1995, 1995-2081, 2081-2168, 2168-2255, 2255-2341, 2341-2428, 2428-2515, or 2515-2602 g, or about 2168 g. In various embodiments, the composition comprises or consists essentially of or consists of Sesame oil at about 384-403, 403-422, 422-442, 442-461, 461-480, 480-499, 499-518, 518-538, 538-557, or 557-576 g, or about 480 g. In various embodiments, the composition comprises or consists essentially of or consists of Cetearyl Alc. & Cetearyl Gluco. at about 96-101, 101-106, 106-110, 110-115, 115-120, 120-125, 125-130, 130-134, 134-139, or 139-144 g, or about 120 g. In various embodiments, the composition comprises or consists essentially of or consists of Cetearyl Alcohol at about 96-101, 101-106, 106-110, 110-115, 115-120, 120-125, 125-130, 130-134, 134-139, or 139-144 g, or about 120 g. In various embodiments, the composition comprises or consists essentially of or consists of Glyceryl & PEG-100 Stearate at about 96-101, 101-106, 106-110, 110-115, 115-120, 120-125, 125-130, 130-134, 134-139, or 139-144 g, or about 120 g. In various embodiments, the composition comprises or consists essentially of or consists of Phospholipids (e.g., PC, PA, PE, and/or PS) at about 96-101, 101-106, 106-110, 110-115, 115-120, 120-125, 125-130, 130-134, 134-139, or 139-144 g, or about 120 g. In various embodiments, the composition comprises or consists essentially of or consists of Menthol at about 67.2-70.6, 70.6-73.9, 73.9-77.3, 77.3-80.6, 80.6-84.0, 84.0-87.4, 87.4-90.7, 90.7-94.1, 94.1-97.4, or 97.4-100.8 g, or about 84.0 g. In various embodiments, the composition comprises or consists essentially of or consists of Glycerin Monolaurate at about 64.0-67.2, 67.2-70.4, 70.4-73.6, 73.6-76.8, 76.8-80.0, 80.0-83.2, 83.2-86.4, 86.4-89.6, 89.6-92.8, or 92.8-96.0 g, or about 80.0 g. In various embodiments, the composition comprises or consists essentially of or consists of Boswellia AKBA at about 64.0-67.2, 67.2-70.4, 70.4-73.6, 73.6-76.8, 76.8-80.0, 80.0-83.2, 83.2-86.4, 86.4-89.6, 89.6-92.8, or 92.8-96.0 g, or about 80.0 g. In various embodiments, the composition comprises or consists essentially of or consists of Propylene Glycol at about 64.0-67.2, 67.2-70.4, 70.4-73.6, 73.6-76.8, 76.8-80.0, 80.0-83.2, 83.2-86.4, 86.4-89.6, 89.6-92.8, or 92.8-96.0 g, or about 80.0 g. In various embodiments, the composition comprises or consists essentially of or consists of Glycerin at about 64.0-67.2, 67.2-70.4, 70.4-73.6, 73.6-76.8, 76.8-80.0, 80.0-83.2, 83.2-86.4, 86.4-89.6, 89.6-92.8, or 92.8-96.0 g, or about 80.0 g. In various embodiments, the composition comprises or consists essentially of or consists of Cetyl Myristoleate at about 32.0-33.6, 33.6-35.2, 35.2-36.8, 36.8-38.4, 38.4-40.0, 40.0-41.6, 41.6-43.2, 43.2-44.8, 44.8-46.4, or 46.4-48.0 g, or about 40.0 g. In various embodiments, the composition comprises or consists essentially of or consists of Cetyl alcohol at about 32.0-33.6, 33.6-35.2, 35.2-36.8, 36.8-38.4, 38.4-40.0, 40.0-41.6, 41.6-43.2, 43.2-44.8, 44.8-46.4, or 46.4-48.0 g, or about 40.0 g. In various embodiments, the composition comprises or consists essentially of or consists of Dimethicone at about 32.0-33.6, 33.6-35.2, 35.2-36.8, 36.8-38.4, 38.4-40.0, 40.0-41.6, 41.6-43.2, 43.2-44.8, 44.8-46.4, or 46.4-48.0 g, or about 40.0 g. In various embodiments, the composition comprises or consists essentially of or consists of Shea Butter at about 32.0-33.6, 33.6-35.2, 35.2-36.8, 36.8-38.4, 38.4-40.0, 40.0-41.6, 41.6-43.2, 43.2-44.8, 44.8-46.4, or 46.4-48.0 g, or about 40.0 g. In various embodiments, the composition comprises or consists essentially of or consists of Vitamin E Acetate at about 32.0-33.6, 33.6-35.2, 35.2-36.8, 36.8-38.4, 38.4-40.0, 40.0-41.6, 41.6-43.2, 43.2-44.8, 44.8-46.4, or 46.4-48.0 g, or about 40.0 g. In various embodiments, the composition comprises or consists essentially of or consists of Lipoic Acid at about 32.0-33.6, 33.6-35.2, 35.2-36.8, 36.8-38.4, 38.4-40.0, 40.0-41.6, 41.6-43.2, 43.2-44.8, 44.8-46.4, or 46.4-48.0 g, or about 40.0 g. In various embodiments, the composition comprises or consists essentially of or consists of Winter green oil at about 32.0-33.6, 33.6-35.2, 35.2-36.8, 36.8-38.4, 38.4-40.0, 40.0-41.6, 41.6-43.2, 43.2-44.8, 44.8-46.4, or 46.4-48.0 g, or about 40.0 g. In various embodiments, the composition comprises or consists essentially of or consists of Aloe 10X at about 32.0-33.6, 33.6-35.2, 35.2-36.8, 36.8-38.4, 38.4-40.0, 40.0-41.6, 41.6-43.2, 43.2-44.8, 44.8-46.4, or 46.4-48.0 g, or about 40.0 g. In various embodiments, the composition comprises or consists essentially of or consists of Polyhexameth. Biguanide at about 32.0-33.6, 33.6-35.2, 35.2-36.8, 36.8-38.4, 38.4-40.0, 40.0-41.6, 41.6-43.2, 43.2-44.8, 44.8-46.4, or 46.4-48.0 g, or about 40.0 g. In various embodiments, the composition comprises or consists essentially of or consists of Cyclopentasiloxane at about 25.6-26.9, 26.9-28.2, 28.2-29.4, 29.4-30.7, 30.7-32.0, 32.0-33.3, 33.3-34.6, 34.6-35.8, 35.8-37.1, or 37.1-38.4 g, or about 32.0 g. In various embodiments, the composition comprises or consists essentially of or consists of Ascorbyl Palmitate at about 16.0-16.8, 16.8-17.6, 17.6-18.4, 18.4-19.2, 19.2-20.0, 20.0-20.8, 20.8-21.6, 21.6-22.4, 22.4-23.2, or 23.2-24.0 g, or about 20.0 g. In various embodiments, the composition comprises or consists essentially of or consists of Ethyl Lauryl Arginate at about 16.0-16.8, 16.8-17.6, 17.6-18.4, 18.4-19.2, 19.2-20.0, 20.0-20.8, 20.8-21.6, 21.6-22.4, 22.4-23.2, or 23.2-24.0 g, or about 20.0 g. In various embodiments, the composition comprises or consists essentially of or consists of Ultrez 20 Carbomer at about 9.6-10.1, 10.1-10.6, 10.6-11.0, 11.0-11.5, 11.5-12.0, 12.0-12.5, 12.5-13.0, 13.0-13.4, 13.4-13.9, or 13.9-14.4 g, or about 12.0 g. In various embodiments, the composition comprises or consists essentially of or consists of BHA at about 6.40-6.72, 6.72-7.04, 7.04-7.36, 7.36-7.68, 7.68-8.00, 8.00-8.32, 8.32-8.64, 8.64-8.96, 8.96-9.28, or 9.28-9.60 g, or about 8.00 g. In various embodiments, the composition comprises or consists essentially of or consists of Dehydro Acetic Acid at about 6.40-6.72, 6.72-7.04, 7.04-7.36, 7.36-7.68, 7.68-8.00, 8.00-8.32, 8.32-8.64, 8.64-8.96, 8.96-9.28, or 9.28-9.60 g, or about 8.00 g. In various embodiments, the composition comprises or consists essentially of or consists of Sesamin Complex at about 3.20-3.36, 3.36-3.52, 3.52-3.68, 3.68-3.84, 3.84-4.00, 4.00-4.16, 4.16-4.32, 4.32-4.48, 4.48-4.64, or 4.64-4.80 g, or about 4.00 g. In various embodiments, the composition comprises or consists essentially of or consists of Vinpocetine at about 3.20-3.36, 3.36-3.52, 3.52-3.68, 3.68-3.84, 3.84-4.00, 4.00-4.16, 4.16-4.32, 4.32-4.48, 4.48-4.64, or 4.64-4.80 g, or about 4.00 g. In some embodiments, the composition comprises or consists essentially of or consists of 1, 2, 3, 4, 5, 6, 7, 8, 9, 10, 11, 12, 13, 14, 15, 16, 17, 18, 19, 20, 21, 22, 23, 24, 25, 26, or 27 of these listed ingredients. In some embodiments, the composition comprises or consists essentially of or consists of at least 1, 2, 3, 4, 5, 6, 7, 8, 9, 10, 11, 12, 13, 14, 15, 16, 17, 18, 19, 20, 21, 22, 23, 24, 25, 26, or 27 of these listed ingredients. In some embodiments, the composition comprises or consists essentially of or consists of all of these listed ingredients. In some embodiments, the composition is intended for topical administration.

In various embodiments, the invention teaches a composition for treating a condition characterized by inflammation. In some embodiments, the condition is neuropathy. In some embodiments, the condition is arthritis. In some non-limiting preferred embodiments, the composition includes vinpocetine and one or more of the following compounds: curcumin, piperine, commiphora mukul extract, boswellia serrata extract, boswellia Acetyl-11-keto-β-boswellic acid (AKBA), and withania somnifera. In some non-limiting preferred embodiments, the composition includes vinpocetine, curcumin and piperine. The composition may further include boswellia serrata extract and/or boswellia Acetyl-11-keto-β-boswellic acid (AKBA). In any of the preceding embodiments, the composition may further include one or more phospholipids selected from the group consisting of phosphatidylcholine, phosphatidic acid, phosphatidylethanolamine, and phosphatidylserine. In any of the preceding embodiments, the composition may further include one or more compounds selected from Vitamin C, Vitamin B1, Benfotiamine, Vitamin B2, Vitamin B3, Vitamin B6, Methylcobalamin B12, L-taurine, alpha-lipoic acid and Folic Acid. In any of the preceding embodiments, the composition may further include Vitamin E and/or Vitamin E Acetate. In any of the preceding embodiments, the composition may further include one or more or all of the following: water, sesame oil, mineral oil, vegetable oil, Cetearyl Alc. & Cetearyl Gluco., Cetearyl Alcohol, Glyceryl & PEG-100 Stearate, Menthol, Glycerin Monolaurate, Propylene Glycol, Glycerin, Cetyl Myristoleate, Cetyl alcohol, Dimethicone, Shea Butter, Winter green oil, Aloe 10X, Polyhexameth. Biguanide, Cyclopentasiloxane, Ascorbyl Palmitate, Ethyl Lauryl Arginate, Ultrez 20 Carbomer, BHA, and Dehydro Acetic Acid. In any of the preceding embodiments, the composition may include glycerin monolaurate. In any of the preceding embodiments, the composition may include cetyl myristoleate. In any of the preceding embodiments, the composition may include L-Taurine.

All values in the ranges provided below are intended to be % weight or volume, as defined herein above. Percentages listed are for non-limiting preferred embodiments of both oral and topical formulations, unless otherwise indicated.

Various compositions for the treatment of arthritis are described above. In some embodiments, the compositions include one or more constituents, as indicated above. In some embodiments, the amount(s) of the individual constituent(s) of the compositions (when a given constituent is present, as specified for each embodiment above) are as follows (indicated in parentheses): vinpocetine (0.1-0.5%), curcumin (10-15%), piperine (1-2%), commiphora mokul extract (9-14%), boswellia Acetyl-11-keto-β-boswellic acid (AKBA) (15-22%), and withania somnifera (12-17%). In some embodiments, boswellia Acetyl-11-keto-β-boswellic acid (AKBA) is included in an amount from 15-22% when the composition is intended/formulated (e.g., by any manner described herein) for oral administration, and 1.5-3% when the composition is intended/formulated (e.g., by any manner described herein) for topical administration. In some embodiments, when present in a composition for treating arthritis (as indicated above), phosphatidylcholine is included in an amount from 10-15% when the composition is intended for oral administration, and 2-5% when the composition is intended for topical administration. In some embodiments, when present in a composition for treating arthritis (as indicated above), alpha-lipoic acid is included in an amount from 1-2% when the composition is intended for topical administration. In some embodiments, when present in a composition for treating arthritis (as indicated above), menthol is included in an amount from 1-5% when the composition is intended for topical administration. In some embodiments, when present in a composition for treating arthritis (as indicated above), Glycerin Monolaurate is included in an amount from 1-9%. In some embodiments, when present in a composition for treating arthritis (as indicated above), Cetyl Myristoleate is included in an amount from 2-6%.

Various compositions for the treatment of neuropathy are described above. In some embodiments, the compositions include one or more constituents, as indicated above. In some embodiments, the amount(s) of the individual constituent(s) of the compositions (when a given constituent is present, as specified for each embodiment above) are as follows (indicated in parentheses): vinpocetine (0.15-0.25%), curcumin (3-5%), piperine (0.3-0.7%), commiphora mokul extract (3-5%), and withania somnifera (7-10%). In some embodiments, when present in a composition for treating neuropathy (as indicated above), phosphatidylcholine is present in an amount from 24-32%. In some embodiments, when present in a composition for treating neuropathy (as indicated above), the following constituents are included within the following ranges (indicated in parentheses): Vitamin C (2.5-4%), Vitamin B1 (0-1%), Benfotiamine (4-6%), Vitamin B2 (0.1-0.3%), Vitamin B3 (1.5-2.5%), Vitamin B6 (0.1-0.2%), Methylcobalamin B12 (0.05-0.1%), L-taurine (18-23%), alpha-lipoic acid (10-16%) and Folic Acid (0.01-0.03%).

In some embodiments, when present in a composition for treating neuropathy (as indicated above), L-Taurine is present in an amount from 18-23%. As indicated above, all percentages listed in the foregoing non-limiting preferred embodiments are by weight or volume, as defined above.

In various embodiments, the compositions according to the invention may be formulated for delivery via any route of administration. “Route of administration” may refer to any administration pathway known in the art, including but not limited to oral, topical, aerosol, nasal, via inhalation, transmucosal, transdermal, parenteral, enteral, or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the compositions can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the compositions can be in the form of aerosol, lotion, cream, gel, ointment, suspensions, solutions or emulsions. Methods for these administrations are known to one skilled in the art.

In some embodiments, a composition as disclosed herein is formulated for oral administration. In some embodiments, a composition as disclosed herein is formulated for topical administration. In some embodiments, a composition as disclosed herein is formulated as a cream for topical delivery. In other embodiments, a composition as disclosed herein is formulated as capsules or tablets for oral delivery. In various embodiments, a capsule or tablet of the oral formulation comprises about 400-450, 450-500, 500-550, 550-600, 600-650, 650-700, 700-750, 750-800, 800-850, or 850-900 mg of a composition as disclosed herein. In various embodiments, a capsule or tablet of the oral formulation is taken together with a meal. In some embodiments, a subject takes 1 capsule or tablet per day. In some embodiments, a subject takes 2 capsules or tablets per day. In some embodiments, a subject takes 3 capsules or tablets per day. In some embodiments, a subject takes 4 capsules or tablets per day. In some embodiments, a subject takes 5 capsules or tablets per day. In some embodiments, a subject takes 6 capsules or tablets per day. In some embodiments, a subject takes 7 capsules or tablets per day. In some embodiments, a subject takes 8 capsules or tablets per day. In some embodiments, a subject takes 9 capsules or tablets per day. In some embodiments, a subject takes 3 capsules or tablets per day. In some embodiments, a subject takes at least 1, 2, 3, 4, 5, 6, 7, 8, or 9 capsules or tablets per day. In some embodiments, a subject takes 10 or more capsules per day.

In various embodiments, the pharmaceutical compositions according to the invention can contain any pharmaceutically acceptable excipient. As used herein, an “excipient” is a natural or synthetic substance formulated alongside the active ingredient of a composition or formula, included for the purpose of bulking-up the composition or formula. Thus, “excipient” is often referred to as “bulking agent”, “filler”, or “diluent”. For a non-limiting example, one or more excipients may be added to a composition described herein and increase the composition's volume or size so that one serving of the composition fits into one capsule or tablet. Also, an “excipient” may confer an enhancement on the active ingredients in the final dosage form, such as facilitating absorption or solubility of the active ingredients. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical compositions are made following the conventional techniques of pharmacy involving dry milling, mixing, and blending for powder forms; milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

Polymers formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The pharmaceutical compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

The pharmaceutical composition according to the invention can also be a bead system for delivering the therapeutic agent to the target cells. For example, pectin/zein hydrogel bead system may be used to deliver Neuregulin-4 or a pharmaceutical equivalent, analog, derivative or a salt thereof, to the target cells in the subject (Yan F. et al., J Clin Invest. 2011 June; 121(6):2242-53).

### Methods of the Invention

In various embodiments, the present invention provides a method for treating neuropathy and/or a neuropathy-related condition and/or arthritis in a subject. The method consists of or consists essentially of or comprises: providing a composition for the treatment of neuropathy and/or a neuropathy-related condition and/or arthritis as described herein; and administering an effective amount of the composition to the subject, thereby treating neuropathy and/or the neuropathy-related condition and/or arthritis in the subject. In various embodiments, one, two, or three, or more compositions are provided. In some embodiments, both oral and topical formulations of various compositions as described herein are provided. In other embodiments, either oral or topical formulations of one or more compositions described herein are provided.

In various embodiments, the subject is a human. In some embodiments, a composition as disclosed herein may be administered at the prevention stage (i.e., when the subject has not developed neuropathy and/or a neuropathy-related condition and/or arthritis) but is likely to or in the process of developing neuropathy and/or a neuropathy-related condition and/or arthritis). In other embodiments, a composition as disclosed herein may be administered at the treatment stage (i.e., when the subject has already developed neuropathy and/or a neuropathy-related condition and/or arthritis). In other embodiments, a composition as disclosed herein may be administered at the maintenance stage (i.e., when the subject has been successfully treated for neuropathy and/or a neuropathy-related condition and/or arthritis).

In accordance with the invention, a composition as disclosed herein may be administered using the appropriate modes of administration, for instance, the modes of administration recommended by the manufacturer. In accordance with the invention, various routes may be utilized to administer a composition as disclosed herein for the claimed methods, including but not limited to oral, topical application, intratumoral, intravascular, intravenous, intraarterial, intramuscular, subcutaneous, intraperitoneal, aerosol, nasal, via inhalation, transmucosal, transdermal, parenteral, implantable pump or reservoir, continuous infusion, enteral application, local application, capsules and/or injections. In various embodiments, the composition is administered topically to the subject, for example, to the area of pain. In various embodiments, the composition is administered orally to the subject. In various embodiments, the composition is administered with a meal to the subject. In various embodiments, the composition is administered orally and topically to the subject. In certain embodiments, the method comprises using both oral and topical formulations of various compositions as disclosed herein to treat the subject.

Typical dosages of an effective amount of a composition as disclosed herein can be in the ranges recommended by the manufacturer where known therapeutic molecules or compounds are used, and also as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models.

In various embodiments, the subject may be administered with a composition as disclosed herein at about 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, or 3400-3500 mg per day, or a combination thereof. In some embodiments, a subject ingests 500-10,000 mg per day of one or more compositions described herein. In some embodiments, a subject ingests about 2576 mg per day of one or more compositions described herein. In some embodiments, a subject ingests 6-111 mg/kg body weight per day of one or more compositions described herein. In some embodiments, a subject ingests 30-50 mg/kg body weight per day of one or more compositions described herein.

In some embodiments, the application teaches a method of treating neuropathy and/or a neuropathy-related condition and/or arthritis by administering to an individual with neuropathy and/or a neuropathy-related condition and/or arthritis one or more compositions described herein, by any means of administration described herein. In some embodiments, the application teaches a method of treating neuropathy and/or a neuropathy-related condition and/or arthritis by providing to an individual with neuropathy and/or a neuropathy-related condition and/or arthritis one or more compositions described herein.

In various embodiments, the composition is administered once, twice, three or more times. In various embodiments, the composition is administered one, two, three, four or five times per day. In various embodiments, the composition is administered 1-5 times per day, 1-7 times per week, 1-9 times per month, or 1-12 times per year. In various embodiments, the composition is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In various embodiments, the composition may be administered once a day (SID/QD), twice a day (BID), three times a day (TID), four times a day (QID), or more, so as to administer an effective amount of a composition as disclosed herein to the subject, where the effective amount is any one or more of the doses described herein.

### Kits of the Invention

In various embodiments, the present invention provides a kit for treating neuropathy and/or a neuropathy-related condition and/or arthritis in a subject. The kit consists of or consists essentially of or comprises: a composition as described herein; and instructions for using the composition to treat neuropathy and/or a neuropathy-related condition and/or arthritis in a subject. In various embodiments, one, two, or three, or more compositions are provided. In some embodiments, both oral and topical formulations of various compositions as described herein are provided.

The kit is an assemblage of materials or components, including at least one of the inventive compositions. In one embodiment, the kit consists of or consists essentially of or comprises a composition described herein.

The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome. Optionally, the kit also contains other useful components, such as, containers, spray bottles or cans, diluents, buffers, pharmaceutically acceptable carriers, syringes, catheters, applicators (for example, applicators of cream, gel or lotion etc.), pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the compositions can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in assays and therapies. As used herein, the term “package” refers to a suitable solid matrix or material such as glass, plastic, paper, foil, and the like, capable of holding the individual kit components. Thus, for example, a package can be a glass vial used to contain suitable quantities of a composition as described herein. The packaging material generally has an external label which indicates the contents and/or purpose of the kit and/or its components.

Many variations and alternative elements have been disclosed in embodiments of the present invention. Still further variations and alternate elements will be apparent to one of skill in the art. Among these variations, without limitation, are the selection of constituent modules for the inventive methods, compositions, kits, and systems, and the various conditions, diseases, and disorders that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### Examples

The following examples are offered for illustrative purposes only, and are not intended to limit the scope of the present invention in any way.

### Example 1: Neuropathy Pain Management System

In various non-limiting examples, the present invention provides a neuropathy and/or arthritis pain management system. This system is designed with expanded requirements for comprehensive care. Results may vary from noticeable improvements in a few weeks to possible permanent relief with ongoing usage. In some examples, the system may include oral capsules of a composition as disclosed herein. In some examples, the system may include topical creams of a composition as disclosed herein. In some examples, the system may include both oral capsules and topical creams of a composition as disclosed herein for the treatment of neuropathy and/or arthritis, and the oral capsules and topical creams may work synergistically. Oral capsules may support and resolve patients' systemic imbalance and possibly provide repair and permanent relief. Topical creams may help patients with quick relief of most neuropathy pain symptoms and/or arthritis pain when applied on the area of pain. Features of topical creams include but are not limited to liposomal delivery, targeted delivery, quickly absorbed, captured in fat layer, released slowly, and for localized cell support from alpha lipoic acid.

In an exemplar treatment regime, patients were instructed to use this neuropathy pain management system for six months. They were told they might start seeing significant benefit by the end of 30 days; however, they were instructed to continue the treatment regimen for six months. For oral capsules, recommended dose is four per day, two in the morning and two in the evening, taken with meals. If a patient missed a dose, he was instructed not to double the dosage at the next scheduled time, but to continue with only two at the next scheduled time. For petite and slim people with body weight less than 120 lbs., total of three capsules could be used at doctors' discretion: two in the morning and one in evening or night. After completion of the six-month treatment regimen, patients could be placed on a reduced dose of two capsules per day, one in the morning and one in the evening, as a preventive measure. The compositions described herein contain potent antioxidant properties and help with the oxidative stress and inflammation. Topical creams were recommended twice a day once in the morning and once in the evening, for example, after shower or bath, and may also be used on as needed basis.

Following this exemplar protocol, nine out of eleven neuropathy patients reported a significant benefit after four months of use, indicating almost complete relief of neuropathy symptoms including pain. Eight of the nine patients reporting significant improvement noticed significant pain relief within one month.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the to ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

What is claimed is:

1. A composition for treating neuropathy and/or arthritis comprising vinpocetine and one or more of the following compounds: curcumin, piperine, commiphora mukul extract, boswellia serrata extract, boswellia Acetyl-11-keto-β-boswellic acid (AKBA), and withania somnifera.

2. The composition of claim 1, wherein the composition comprises vinpocetine, curcumin and piperine.

3. The composition of claim 2, further comprising boswellia serrata extract.

4. The composition of claim 1 further comprising one or more phospholipids selected from the group consisting of phosphatidylcholine, phosphatidic acid, phosphatidylethanolamine, and phosphatidylserine.

5. The composition of claim 1, further comprising one or more compound selected from the group consisting of Vitamin C, Vitamin B1, Benfotiamine, Vitamin B2, Vitamin B3, Vitamin B6, Methylcobalamin B12, L-taurine, alpha-lipoic acid and Folic Acid.

6. The composition of claim 5, further comprising Vitamin E and/or Vitamin E Acetate.

7. The composition of claim 1, further comprising one or more of the following: water, sesame oil, mineral oil, vegetable oil, Cetearyl Alc. & Cetearyl Gluco., Cetearyl Alcohol, Glyceryl & PEG-100 Stearate, Menthol, Glycerin Monolaurate, Propylene Glycol, Glycerin, Cetyl Myristoleate, Cetyl alcohol, Dimethicone, Shea Butter, Winter green oil, Aloe 10X, Polyhexameth. Biguanide, Cyclopentasiloxane, Ascorbyl Palmitate, Ethyl Lauryl Arginate, Ultrez 20 Carbomer, BHA, and Dehydro Acetic Acid.

8. The composition of claim 7, wherein the composition comprises glycerin monolaurate

9. The composition of claim 7, wherein the composition comprises cetyl myristoleate

10. The composition of claim 1, wherein the composition comprises L-Taurine.

11. A method for treating a condition associated with inflammation, comprising:
providing a composition comprising vinpocetine and one or more of the following compounds: curcumin, piperine, commiphora mokul extract, boswellia serrata extract, boswellia Acetyl-11-keto-β-boswellic acid (AKBA), and withania somnifera; and
administering an effective amount of the composition to the subject, thereby treating the condition associated with inflammation.

12. The method of claim 11, wherein the composition comprises vinpocetine, curcumin and piperine.

13. The method of claim 12, wherein the composition further comprises boswellia serrata extract.

14. The method of claim 11, wherein the composition further comprises one or more phospholipids selected from the group consisting of phosphatidylcholine, phosphatidic acid, phosphatidylethanolamine, and phosphatidylserine.

15. The method of claim 11, wherein the composition further comprises one or more compound selected from the group consisting of Vitamin C, Vitamin B1, Benfotiamine, Vitamin B2, Vitamin B3, Vitamin B6, Methylcobalamin B12, L-taurine, alpha-lipoic acid and Folic Acid.

16. The method of claim 15, wherein the composition further comprises Vitamin E and/or Vitamin E Acetate.

17. The method of claim 11, wherein the composition further comprises one or more of the following: water, sesame oil, mineral oil, vegetable oil, Cetearyl Alc. & Cetearyl Gluco., Cetearyl Alcohol, Glyceryl & PEG-100 Stearate, Menthol, Glycerin Monolaurate, Propylene Glycol, Glycerin, Cetyl Myristoleate, Cetyl alcohol, Dimethicone, Shea Butter, Winter green oil, Aloe 10X, Polyhexameth. Biguanide, Cyclopentasiloxane, Ascorbyl Palmitate, Ethyl Lauryl Arginate, Ultrez 20 Carbomer, BHA, and Dehydro Acetic Acid.

18. The method of claim 17, wherein the composition comprises glycerin monolaurate.

19. The method of claim 7, wherein the composition comprises cetyl myristoleate.

20. The method of claim 11, wherein the composition comprises L-Taurine.

21. The method of claim 11, wherein the conditions associated with inflammation is neuropathy.

22. The method of claim 11, wherein the condition associated with inflammation is arthritis.

23. The method of claim 11, wherein the subject is a human.

24. The method of claim 11, wherein the composition is administered orally to the subject.

25. The method of claim 11, wherein the composition is administered topically to the subject.

26. A kit for treating a condition associated with inflammation in a subject, comprising:
a composition of claim 1; and
instructions for using the composition to treat a condition associated with inflammation.

27. The kit of claim 26, wherein the condition is neuropathy.

28. The kit of claim 26, wherein the condition is arthritis.
